SEQUENCE LISTING (16 sequences)

SEQ ID NO: 1 - protein, 108 aa, synthetic construct
EIVLTQSPGTLSLSPGERATLSCRASQRVSSSYLAWYQQKPGQAPRLLIYDASSRATGIPDRFSGSGSGTDFTLTISRLEPEDFAVYYCQQYGSLPWTFGQGTKVEIK

SEQ ID NO: 2 - protein, 121 aa, synthetic construct
EVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSS

SEQ ID NO: 3 - protein, 10 aa, synthetic construct
GFTFSRYWMS

SEQ ID NO: 4 - protein, 17 aa, synthetic construct
NIKQDGSEKYYVDSVKG

SEQ ID NO: 5 - protein, 12 aa, synthetic construct
EGGWFGELAFDY

SEQ ID NO: 6 - protein, 12 aa, synthetic construct
RASQRVSSSYLA

SEQ ID NO: 7 - protein, 7 aa, synthetic construct
DASSRAT

SEQ ID NO: 8 - protein, 9 aa, synthetic construct
QQYGSLPWT

SEQ ID NO: 9 - protein, 139 aa, synthetic construct
PSSLSASVGDRVTITCRASQSINSYLDWYQQKPGKAPKLLIYAASSLQSGVPSRFSGSGSGTDFTLTISSLQPEDFATYYCQQYYSTPFTFGPGTKVEIKRTVAAPSVFIFPPSDEQLKSGTASVVCLLNNFYPREAKV

SEQ ID NO: 10 - protein, 167 aa, synthetic construct
GVVQPGRSLRLSCAASGFTFSSYGMHWVRQAPGKGLEWVAVIWYDGSNKYYADSVKGRFTISRDNSKNTLYLQMNSLRAEDTAVYYCARDPRGATLYYYYYGMDVWGQGTTVTVSSASTKGPSVFPLAPCSRSTSESTAALGCLVKDYFPEPVTVSWNSGALTSGVH

SEQ ID NO: 11 - protein, 10 aa, synthetic construct
GFTFSSYGMH

SEQ ID NO: 12 - protein, 15 aa, synthetic construct
VIWYDGSNKYYADSV

SEQ ID NO: 13 - protein, 16 aa, synthetic construct
DPRGATLYYYYYGMDV

SEQ ID NO: 14 - protein, 11 aa, synthetic construct
RASQSINSYLD

SEQ ID NO: 15 - protein, 7 aa, synthetic construct
AASSLQS

SEQ ID NO: 16 - protein, 9 aa, synthetic construct
QQYYSTPFT

(19) United States
(12) Patent Application Publication
(10) Pub. No.: US 2026/0077040 A1
(43) Pub. Date: Mar. 19, 2026

(54) COMPOSITIONS AND METHODS FOR TREATING ADVANCED SOLID TUMORS

(71) Applicant: ASTRAZENECA AB, Södertälje (SE)

(72) Inventor: Haiyi JIANG, Wihnington, DE (US)

(21) Appl. No.: 19/108,552

(22) PCT Filed: Sep. 8, 2023
(86) PCT No.: PCT/EP2023/074693

§ 371 Date: Mar. 4, 2025

Related U.S. Application Data
(60) Provisional application No. 63/405,139, filed on Sep. 9, 2022.

(51) Int. Cl. A61K 39/395; A61K 31/7048; A61K 33/243; A61K 39/00; A61P 35/00

(52) U.S. Cl. CPC A61K 39/3955; A61K 31/7048; A61K 33/243; A61P 35/00; A61K 2039/507; A61K 2039/54; A61K 2039/545

(57) ABSTRACT

Disclosed are methods for treating solid tumors with an anti-PD-L1 antibody and an anti-CTLA-4 antibody concurrently with chemoradiation therapy (cCRT).

### FIELD OF THE INVENTION

This disclosure relates to methods for treating advanced solid tumors with an anti-PD-L1 antibody concurrently with chemoradiation therapy (cCRT) and optionally an anti-CTLA-4 antibody.

### BACKGROUND

Cancer continues to be a major global health burden. In the United States, it is the second most common cause of death after heart disease, accounting for nearly 1 in every 4 deaths. The 5-year survival rate for all cancers diagnosed between 1999 and 2006 is 68%, which is 18% higher than the rate reported between 1975 and 1977, likely reflecting progress in diagnosing certain cancers earlier and improvements in treatment.

About 12.7 million cancer cases and 7.6 million cancer deaths are estimated to have occurred in 2008 worldwide. Most often these cancers are diagnosed at an advanced or metastatic stage where the life expectancy is very poor. Despite recent advances in chemotherapeutics and in the understanding of the molecular biology of cancer, there has been limited progress in the therapeutic options for advanced and metastatic disease. The poor prognosis reflects the limited efficacy of the treatment options available, highlighting the need for the development of newer therapeutic options.

### SUMMARY

The disclosure generally relates to methods for treating advanced solid tumors with an anti-PD-L1 antibody concurrently with chemoradiation therapy (cCRT) and optionally an anti-CTLA-4 antibody.

In one aspect, the disclosure herein provides a method of increasing the overall response rate (ORR) in a patient with small-cell lung cancer (SCLC), comprising concurrently treating the patient with a human anti-PD-L1 antibody, an anti-CTLA-4 antibody, and chemoradiation therapy.

In another aspect, the disclosure herein provides a method of increasing the disease control rate (DCR) in a patient with SCLC, comprising concurrently treating the patient with a human anti-PD-L1 antibody, an anti-CTLA-4 antibody, and chemoradiation therapy.

In a further aspect, the disclosure herein provides a method of treating a patient with SCLC, comprising concurrently treating the patient with a human anti-PD-L1 antibody, an anti-CTLA-4 antibody, and chemoradiation therapy.

In a further aspect, the disclosure herein provides a method of increasing the overall response rate (ORR) in a patient with squamous cell carcinoma of the head and neck (HNSCC), comprising concurrently treating the patient with a human anti-PD-L1 antibody and chemoradiation therapy.

In a further aspect, the disclosure herein provides a method of increasing the disease control rate (DCR) in a patient with HNSCC, comprising concurrently treating the patient with a human anti-PD-L1 antibody and chemoradiation therapy.

In a further aspect, the disclosure herein provides a method of treating a patient with HNSCC, comprising concurrently treating the patient with a human anti-PD-L1 antibody and chemoradiation therapy.

In a further aspect, the disclosure herein provides a combination comprising a human anti-PD-L1 antibody, an anti-CTLA-4 antibody, and concurrent chemoradiation therapy for use in a method of increasing the overall response rate (ORR) in a patient with SCLC.

In a further aspect, the disclosure herein provides a combination comprising a human anti-PD-L1 antibody, an anti-CTLA-4 antibody, and concurrent chemoradiation therapy for use in a method of increasing the disease control rate (DCR) in a patient with SCLC.

In a further aspect, the disclosure herein provides a combination comprising a human anti-PD-L1 antibody, an anti-CTLA-4 antibody, and concurrent chemoradiation therapy for use in the treatment of SCLC.

In a further aspect, the disclosure herein provides a combination comprising a human anti-PD-L1 antibody and concurrent chemoradiation therapy for use in a method of increasing the overall response rate (ORR) in a patient with HNSCC.

In a further aspect, the disclosure herein provides a combination comprising a human anti-PD-L1 antibody and concurrent chemoradiation therapy for use in a method increasing the disease control rate (DCR) in a patient with HNSCC.

In a further aspect, the disclosure herein provides a combination comprising a human anti-PD-L1 antibody and concurrent chemoradiation therapy for use in the treatment of HNSCC.

In a further aspect, the disclosure herein provides the use of a combination comprising a human anti-PD-L1 antibody, an anti-CTLA-4 antibody, and concurrent chemoradiation therapy in the manufacture of a medicament for use in a method of increasing the overall response rate (ORR) in a patient with SCLC.

In a further aspect, the disclosure herein provides the use of a combination comprising a human anti-PD-L1 antibody, an anti-CTLA-4 antibody, and concurrent chemoradiation therapy in the manufacture of a medicament for use in a method of increasing the disease control rate (DCR) in a patient with SCLC.

In a further aspect, the disclosure herein provides the use of a combination comprising a human anti-PD-L1 antibody, an anti-CTLA-4 antibody, and concurrent chemoradiation therapy in the manufacture of a medicament for use in the treatment of SCLC.

In a further aspect, the disclosure herein provides the use of a combination comprising a human anti-PD-L1 antibody and concurrent chemoradiation therapy in the manufacture of a medicament for use in a method of increasing the overall response rate (ORR) in a patient with HNSCC.

In a further aspect, the disclosure herein provides the use of a combination comprising a human anti-PD-L1 antibody and concurrent chemoradiation therapy in the manufacture of a medicament for use in a method of increasing the disease control rate (DCR) in a patient with HNSCC.

In a further aspect, the disclosure herein provides the use of a combination comprising a human anti-PD-L1 antibody and concurrent chemoradiation therapy in the manufacture of a medicament for use in the treatment of HNSCC.

### BRIEF DESCRIPTION OF THE DRAWINGS

FIG. 1 shows the general study design for the methods disclosed herein.

FIG. 2 shows a detailed study flowchart for the squamous cell carcinoma of the head and neck (HNSCC) cohort described in Example 1.

FIG. 3 shows a detailed study flowchart for the non-small-cell lung cancer (NSCLC) cohort described in Example 1.

FIG. 4 shows a detailed study flowchart for the small-cell lung cancer (SCLC) cohort described in Example 1.

FIG. 5 shows the disease free survival in HNSCC.

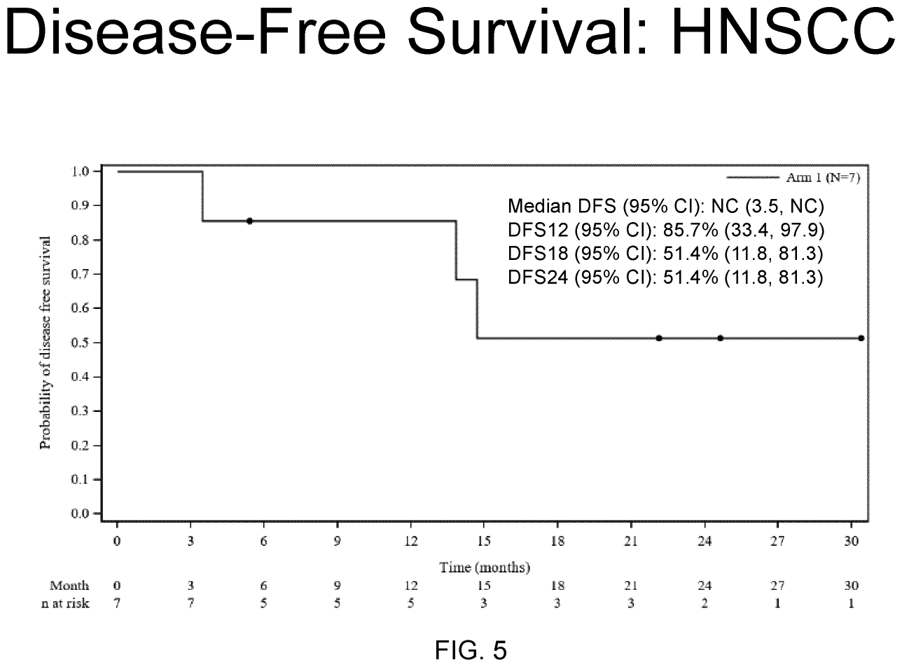

FIG. 6 shows overall survival in HNSCC.

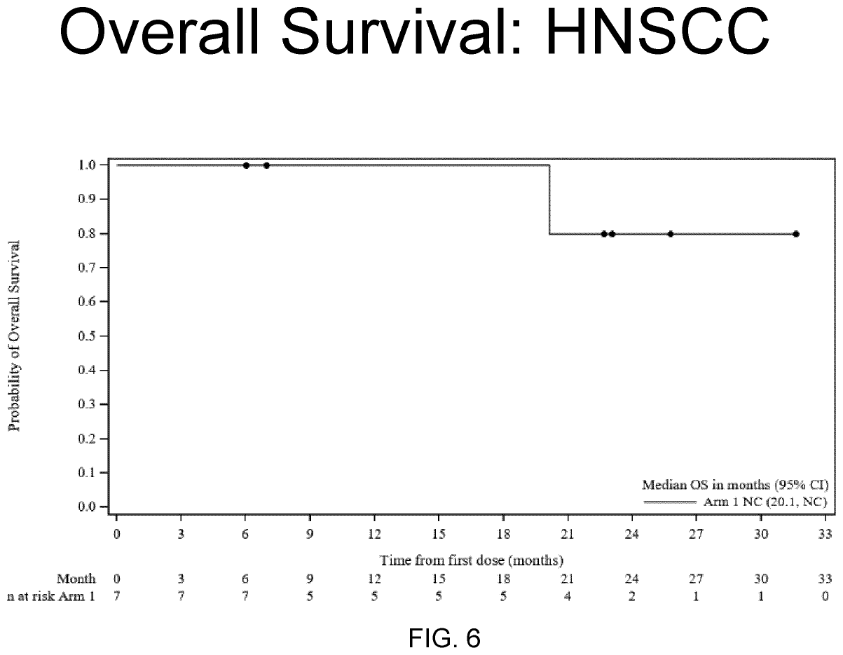

FIG. 7 shows progression-free survival (PFS) and overall survival (OS) in SCLC in arms 1 and 2 of the study.

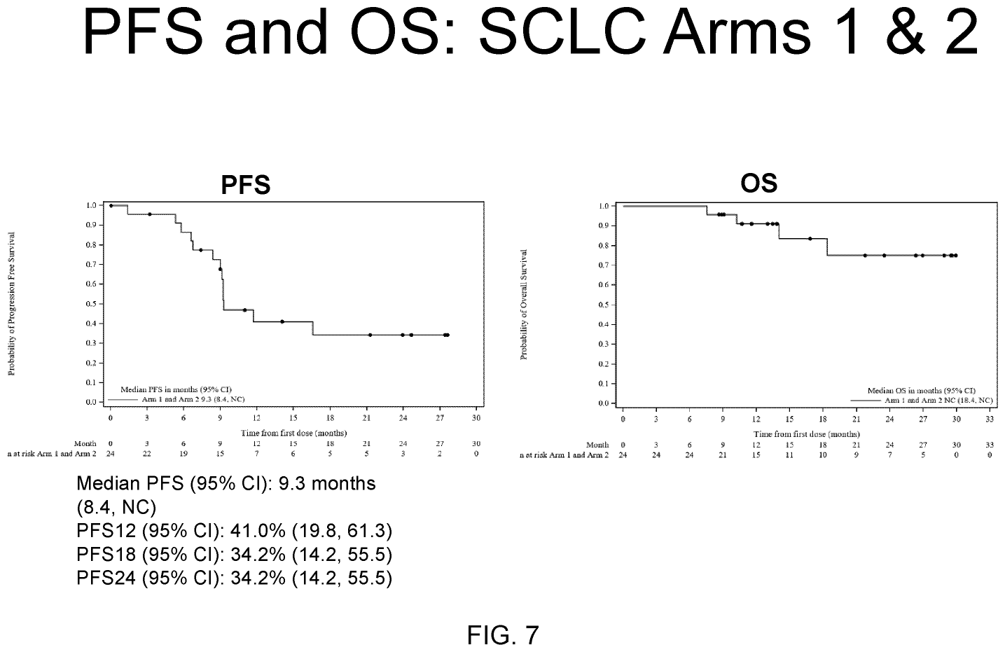

FIG. 8 shows progression-free survival (PFS) and overall survival (OS) in SCLC in arms 1 and 2 of the study by treatment arm.

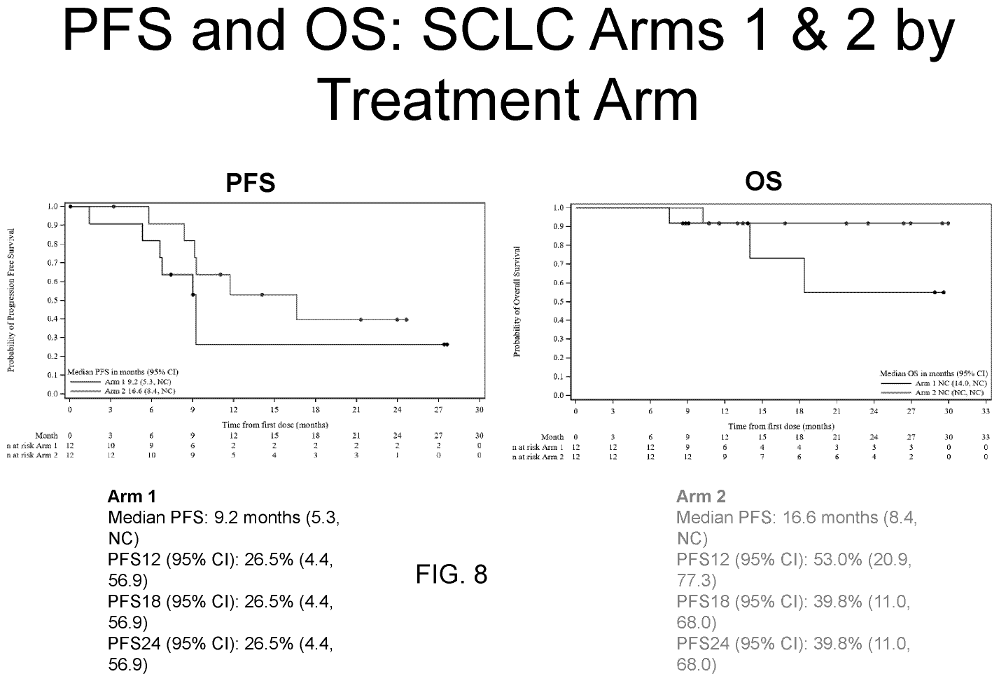

FIG. 9 shows progression-free survival (PFS) and overall survival (OS) in SCLC in arms 3 and 4 of the study.

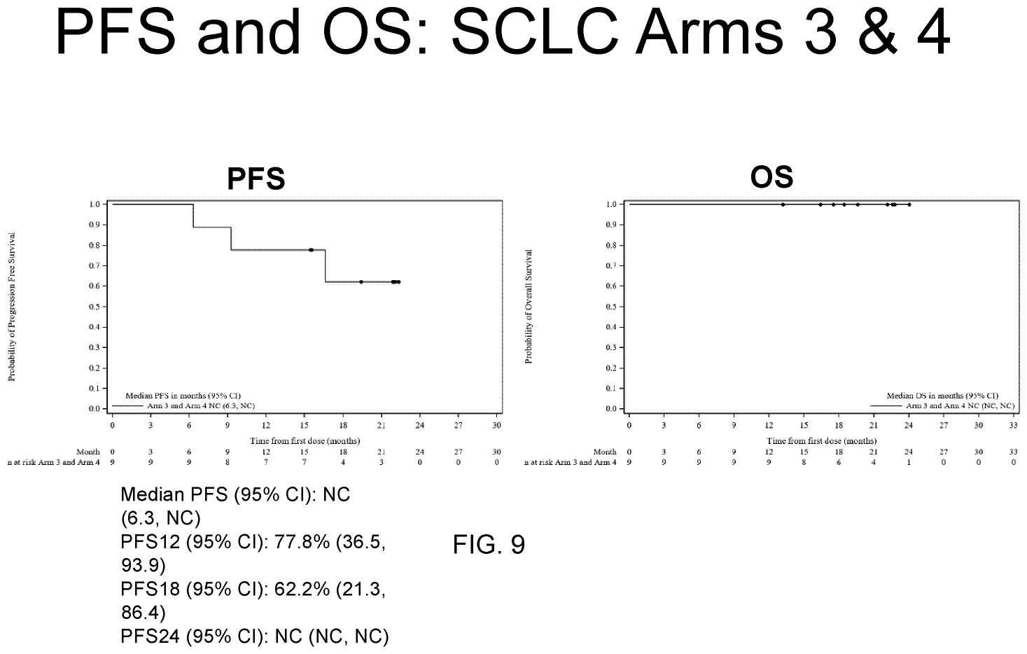

FIG. 10 shows progression-free survival (PFS) and overall survival (OS) in SCLC in arms 3 and 4 of the study by treatment arm.

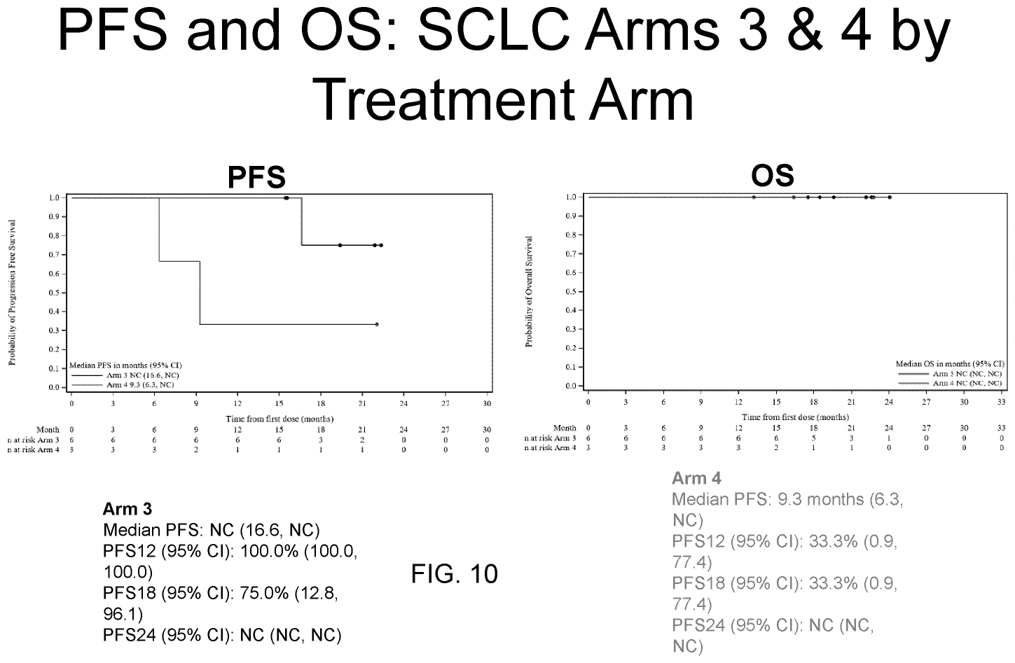

### DETAILED DESCRIPTION

Unless defined otherwise, all technical and scientific terms used herein have the meaning commonly understood by a person skilled in the art to which this disclosure belongs. The following references provide one of skill with a general definition of many of the terms used in this disclosure: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

In this disclosure, “comprises,” “comprising,” “containing,” and “having,” and the like can have the meaning ascribed to them in U.S. patent law and can mean “includes,” “including,” and the like; “consisting essentially of” or “consists essentially” likewise have the meaning ascribed in U.S. patent law and are open-ended, allowing for the presence of more than that which is recited so long as basic or novel characteristics of that which is recited are not changed by the presence of more than that which is recited, but excludes prior art aspects.

Unless specifically stated or obvious from context, the term “or,” as used herein, is understood to be inclusive. Unless specifically stated or obvious from context, the terms “a,” “an,” and “the,” as used herein, are understood to be singular or plural.

Unless specifically stated or obvious from context, the term “about,” as used herein, is understood as meaning within a range of normal tolerance in the art, for example within 2 standard deviations of the mean. “About” can be understood as meaning within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

Any compositions or methods provided herein can be combined with one or more of any of the other compositions and methods provided herein.

Ranges provided herein are understood to be shorthand for all of the values within the range. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

The term “antibody,” as used herein, refers to an immunoglobulin or a fragment or a derivative thereof, and encompasses any polypeptide comprising an antigen-binding site, regardless of whether it is produced in vitro or in vivo. The term includes, but is not limited to, polyclonal, monoclonal, monospecific, polyspecific, non-specific, humanized, single-chain, chimeric, synthetic, recombinant, hybrid, mutated, and grafted antibodies. Unless otherwise modified by the term “intact,” as in “intact antibodies,” for the purposes of this disclosure, the term “antibody” also includes antibody fragments such as Fab, F(ab′)₂, Fv, scFv, Fd, dAb, and other antibody fragments that retain antigen-binding function, i.e., the ability to bind PD-L1 specifically. Typically, such fragments would comprise an antigen-binding domain.

The terms “antigen-binding domain,” “antigen-binding fragment,” and “binding fragment,” as used herein, refer to a part of an antibody molecule that comprises amino acids responsible for the specific binding between the antibody and the antigen. In some instances, where an antigen is large, the antigen-binding domain may only bind to a part of the antigen. A portion of the antigen molecule that is responsible for specific interactions with the antigen-binding domain is referred to as “epitope” or “antigenic determinant.” An antigen-binding domain typically comprises an antibody light chain variable region (V_{L}) and an antibody heavy chain variable region (V_{H}); however, it does not necessarily have to comprise both. For example, a so-called Fd antibody fragment consists only of a V_{H} domain, but still retains some antigen-binding function of the intact antibody.

In some embodiments disclosed herein is a method of increasing the overall response rate (ORR) in a patient with small-cell lung cancer (SCLC), the method comprising concurrently treating the patient with a human anti-PD-L1 antibody, an anti-CTLA-4 antibody, and chemoradiation therapy.

In some embodiments disclosed herein is a method of increasing the disease control rate (DCR) in a patient with SCLC, the method comprising concurrently treating the patient with a human anti-PD-L1 antibody, an anti-CTLA-4 antibody, and chemoradiation therapy.

In some embodiments disclosed herein is a method of treating a patient with SCLC, the method comprising concurrently treating the patient with a human anti-PD-L1 antibody, an anti-CTLA-4 antibody, and chemoradiation therapy.

In some embodiments disclosed herein is a method of increasing the overall response rate (ORR) in a patient with squamous cell carcinoma of the head and neck (HNSCC), the method comprising concurrently treating the patient with a human anti-PD-L1 antibody and chemoradiation therapy.

In some embodiments disclosed herein is a method of increasing the disease control rate (DCR) in a patient with HNSCC, the method comprising concurrently treating the patient with a human anti-PD-L1 antibody and chemoradiation therapy

In some embodiments disclosed herein is a method of treating a patient with HNSCC, the method comprising concurrently treating the patient with a human anti-PD-L1 antibody and chemoradiation therapy.

In some embodiments, the anti-PD-L1 antibody is durvalumab, avelumab, atezolizumab, or sugemalimab. In some embodiments, the anti-PD-L1 antibody is durvalumab. Durvalumab (MEDI4736, Imfinzi®) is a human monoclonal antibody directed against human PD-L1 that is capable of blocking the binding of PD-L1 to both the PD1 and CD80 receptors. Disclosure related to durvalumab can be found in U.S. Pat. Nos. 8,779,108 and 9,493,565, which are incorporated herein by reference.

Durvalumab for use in the methods, compositions, and combinations provided herein comprises a heavy chain and a light chain or a heavy chain variable region and a light chain variable region. In some embodiments, durvalumab for use in the methods, compositions, and combinations provided herein comprises a light chain variable region comprising the amino acid sequence of SEQ ID NO: 1 and a heavy chain variable region comprising the amino acid sequence of SEQ ID NO: 2. In some embodiments, durvalumab for use in the methods, compositions, and combinations provided herein comprises a heavy chain variable region and a light chain variable region, wherein the heavy chain variable region comprises the Kabat-defined CDR1, CDR2, and CDR3 sequences of SEQ ID NOs: 3-5, and wherein the light chain variable region comprises the Kabat-defined CDR1, CDR2, and CDR3 sequences of SEQ ID NOs: 6-8. Those of ordinary skill in the art would easily be able to identify Chothia-defined, Abm-defined, or other CDR definitions known to those of ordinary skill in the art. In some embodiments, durvalumab for use in the methods, compositions, and combinations provided herein comprises the variable heavy chain and variable light chain CDR sequences of the 2.14H9OPT antibody as disclosed in U.S. Pat. Nos. 8,779,108 and 9,493,565, which are incorporated herein by reference in their entirety.

Durvalumab light chain (LC) variable region:
(SEQ ID NO: 1)
EIVLTQSPGTLSLSPGERATLSCRASQRVSSSYLAWYQQKPGQAPRLLI
YDASSRATGIPDRFSGSGSGTDFTLTISRLEPEDFAVYYCQQYGSLPWT
FGQGTKVEIK
Durvalumab heavy chain (HC) variable region:
(SEQ ID NO: 2)
EVQLVESGGGLVQPGGSLRLSCAASGFTFSRYWMSWVRQAPGKGLEWVA
NIKQDGSEKYYVDSVKGRFTISRDNAKNSLYLQMNSLRAEDTAVYYCAR
EGGWFGELAFDYWGQGTLVTVSS
Durvalumab heavy chain CDRs:
HC-CDR1:
(SEQ ID NO: 3)
GFTFSRYWMS
HC-CDR2:
(SEQ ID NO: 4)
NIKQDGSEKYYVDSVKG
HC-CDR3:
SEQ ID NO: 5)
EGGWFGELAFDY
Durvalumab, light chain CDRs:
LC-CDR1:
(SEQ ID NO: 6)
RASQRVSSSYLA
LC-CDR2:
(SEQ ID NO: 7)
DASSRAT
LC-CDR3:
(SEQ ID NO: 8)
QQYGSLPWT

In some embodiments, the anti-CTLA-4 antibody is tremelimumab. Tremelimumab for use in the methods, compositions, and combinations provided herein comprises a heavy chain and a light chain or a heavy chain variable region and a light chain variable region. In some embodiments, tremelimumab for use in the methods, compositions, and combinations provided herein comprises a light chain variable region comprising the amino acid sequence of SEQ ID NO: 9 and a heavy chain variable region comprising the amino acid sequence of SEQ ID NO: 10. In some embodiments, tremelimumab for use in the methods, compositions, and combinations provided herein comprises a heavy chain variable region and a light chain variable region, wherein the heavy chain variable region comprises the Kabat-defined CDR1, CDR2, and CDR3 sequences of SEQ ID NOs: 11-13, and wherein the light chain variable region comprises the Kabat-defined CDR1, CDR2, and CDR3 sequences of SEQ ID NOs: 14-16. Those of ordinary skill in the art would easily be able to identify Chothia-defined, Abm-defined, or other CDR definitions known to those of ordinary skill in the art. In some embodiments, tremelimumab for use in the methods, compositions, and combinations provided herein comprises or the variable heavy chain and variable light chain CDR sequences of the 11.2.1 antibody as disclosed in U.S. Pat. No. 6,682,736, which is incorporated herein by reference in its entirety.

Tremelimumab light chain (LC) variable region:
(SEQ ID NO: 9)
PSSLSASVGDRVTITCRASQSINSYLDWYQQKPGKAPKLLIYAASSLQS
GVPSRFSGSGSGTDFTLTISSLQPEDFATYYCQQYYSTPFTFGPGTKVE
IKRTVAAPSVFIFPPSDEQLKSGTASVVCLLNNFYPREAKV
Tremelimumab heavy chain (HC) variable region:
(SEQ ID NO: 10)
GVVQPGRSLRLSCAASGFTFSSYGMHWVRQAPGKGLEWVAVIWYDGSNK
YYADSVKGRFTISRDNSKNTLYLQMNSLRAEDTAVYYCARDPRGATLYY
YYYGMDVWGQGTTVTVSSASTKGPSVFPLAPCSRSTSESTAALGCLVKD
YFPEPVTVSWNSGALTSGVH
Tremelimumab heavy chain CDRs
HC-CDR1:
(SEQ ID NO: 11)
GFTFSSYGMH
HC-CDR2:
(SEQ ID NO: 12)
VIWYDGSNKYYADSV
HC-CDR3:
(SEQ ID NO: 13)
DPRGATLYYYYYGMDV
Tremelimumab light chain CDR1s
LC-CDR1:
(SEQ ID NO: 14)
RASQSINSYLD
LC-CDR2:
(SEQ ID NO: 15)
AASSLQS
LC-CDR3:
(SEQ ID NO: 16)
QQYYSTPFT

Binding fragments of an antibody are produced by recombinant DNA techniques, or by enzymatic or chemical cleavage of intact antibodies. Binding fragments include Fab, Fab′, F(ab′)2, Fv, and single-chain antibodies. An antibody other than a “bispecific” or “bifunctional” antibody is understood as an antibody in which each of its binding sites is identical. Digestion of antibodies with the enzyme papain results in two identical antigen-binding fragments, known also as “Fab” fragments, and a “Fc” fragment, having no antigen-binding activity but having the ability to crystallize. Digestion of antibodies with the enzyme pepsin results in a F(ab′)2 fragment in which the two arms of the antibody molecule remain linked and comprise two-antigen binding sites. The F(ab′)2 fragment has the ability to crosslink antigen. The term “Fv,” as used herein, refers to the minimum fragment of an antibody that retains both antigen-recognition and antigen-binding sites. The term “Fab,” as used herein, refers to a fragment of an antibody that comprises the constant domain of the light chain and the CHI domain of the heavy chain.

In some embodiments, the methods, compositions, and combinations disclosed herein relate to treating a subject for a tumor disorder and/or a cancer disorder. In some embodiments, the tumor is an advanced solid tumor. In some embodiments, the cancer is selected from small-cell lung cancer, squamous cell carcinoma of the head and neck and/or non-small-cell lung cancer.

In particular embodiments, the small-cell lung cancer is limited stage.

The term “solid tumor,” as used herein, refers to an abnormal mass of tissue that normally does not contain cysts or liquid areas.

The terms “treatment” or “treat,” as used herein, refer to both therapeutic treatment and prophylactic or preventative measures. Those in need of treatment include subjects having cancer as well as those prone to having cancer or those in which cancer is to be prevented. In some embodiments, the methods, compositions, and combinations disclosed herein can be used for the treatment of cancer. In other embodiments, those in need of treatment include subjects having a tumor as well as those prone to have a tumor or those in which a tumor is to be prevented. In certain embodiments, the methods, compositions, and combinations disclosed herein can be used for the treatment of tumors. In other embodiments, treatment of a tumor includes inhibiting tumor growth, promoting tumor reduction, or both inhibiting tumor growth and promoting tumor reduction.

The terms “administration” or “administering,” as used herein, refer to providing, contacting, and/or delivering a compound or compounds by any appropriate route to achieve the desired effect. Administration may include, but is not limited to, oral, sublingual, parenteral (e.g., intravenous, subcutaneous, intracutaneous, intramuscular, intraarticular, intraarterial, intrasynovial, intrasternal, intrathecal, intralesional, or intracranial injection), transdermal, topical, buccal, rectal, vaginal, nasal, ophthalmic, via inhalation, and implants.

Provided herein are methods of treating a tumor in a subject in need thereof, comprising administering to the subject an anti-PD-L1 antibody at a dose of between 5 mg/kg to 20 mg/kg. In some embodiments, provided herein is a method of treating tumor in a subject in need thereof, comprising administering to the subject an anti-PD-L1 antibody or an antigen-binding fragment thereof at a flat dose of between 1000 mg to 2000 mg. In some embodiments, provided herein is a method of treating tumor in a subject in need thereof, comprising administering to the subject an anti-PD-L1 antibody or an antigen-binding fragment thereof at a flat dose of 1500 mg.

In particular embodiments, the subject is administered one or more flat doses. In some embodiments, provided herein is a method of treating tumor in a subject in need thereof, comprising administering to the subject an anti-PD-L1 antibody, wherein the dose is 1000 mg, 1100 mg, 1200 mg, 1300 mg, 1400 mg, 1500 mg, 1600 mg, 1700 mg, 1800 mg, 1900 mg or 2000 mg. In some embodiments, the subject is administered one or more flat doses of the anti-PD-L1 antibody wherein the dose is 1500 mg.

In particular embodiments, the anti-PD-L1 antibody is administered over a two-week treatment period, over a four-week treatment period, over a six-week treatment period, over an eight-week treatment period, over a twelve-week treatment period, over a twenty-four-week treatment period, or over a one-year or more treatment period. In certain embodiments, the anti-PD-L1 antibody is administered over a three-week treatment period, over a six-week treatment period, over a nine-week treatment period, over a twelve-week treatment period, over a twenty-four-week treatment period, or over a one-year or more treatment period. In certain embodiments, the anti-PD-L1 antibody administered over a two-month treatment period, over a four-month treatment period, or over a six-month or more treatment period.

The terms “co-administered,” “in combination,” or “combination therapy,” as used herein, refer to simultaneous or sequential administration of multiple compounds or agents. A first compound or agent may be administered before, concurrently with, or after administration of a second compound or agent. The first compound or agent and the second compound or agent may be simultaneously or sequentially administered on the same day, or may be sequentially administered within 1 day, 2 days, 3 days, 4 days, 5 days, 6 days, 1 week, 2 weeks, 3 weeks, or 1 month of each other. In some embodiments, compounds or agents are co-administered during the period in which each of the compounds or agents are exerting at least some physiological effect and/or has remaining efficacy.

In some embodiments, the anti-PD-L1 antibody is administered concurrently with chemoradiation therapy. The term “concurrently,” as used herein, refers to the administration of the anti-PD-L1 antibody and administration of chemoradiation therapy within about three days of each other. In some embodiments, the anti-PD-L1 antibody is administered within about two days of chemoradiation therapy. In some embodiments, the anti-PD-L1 antibody is administered within about one day of chemoradiation therapy. In some embodiments, the anti-PD-L1 antibody is administered on Cycle 1 Day 1 of chemoradiation therapy.

In some embodiments, the anti-PD-L1 antibody is administered concurrently with an anti-CTLA-4 antibody, and chemoradiation therapy. In particular embodiments, the anti-PD-L1 antibody and the anti-CTLA-4 antibody are administered simultaneously, separately, or sequentially. In some embodiments, the anti-PD-L1 antibody and the anti-CTLA-4 antibody are administered within about three days of the administration of the chemoradiation therapy. In some embodiments, the anti-PD-L1 antibody and the anti-CTLA-4 antibody are administered within about two days of the administration of the chemoradiation therapy. In some embodiments, the anti-PD-L1 antibody and the anti-CTLA-4 antibody are administered within about one day of the administration of the chemoradiation therapy. In some embodiments, the anti-PD-L1 antibody and the anti-CTLA-4 antibody are administered on Cycle 1 Day 1 of chemoradiation therapy.

In some embodiments, the anti-CTLA-4 antibody is administered wherein the dose is 1 mg/kg, 2 mg/kg, 3 mg/kg, 4 mg/kg, or 5 mg/kg. In particular embodiments, the subject is administered one or more flat doses of the anti-CTLA-4 antibody wherein the dose is 75 mg, 100 mg, 125 mg, 150 mg, 175 mg, 200 mg, 225 mg, 250 mg, 275 mg, 300 mg. 325 mg, 350 mg, 375 mg, 400 mg, 425 mg, 450 mg, 475 mg, or 500 mg.

In particular embodiments, the anti-CTLA-4 antibody thereof is administered every week, every two weeks, every four weeks, every six weeks, every eight weeks, every ten weeks, or every twelve weeks.

In particular embodiments, the anti-CTLA-4 antibody is administered over a two-week treatment period, over a four-week treatment period, over a six-week treatment period, over an eight-week treatment period, over a twelve-week treatment period, over a twenty-four-week treatment period, or over a one-year or more treatment period. In certain embodiments, the anti-CTLA-4 antibody is administered over a three-week treatment period, over a six-week treatment period, over a nine-week treatment period, over a twelve-week treatment period, over a twenty-four-week treatment period, or over a one-year or more treatment period. In certain embodiments, the anti-CTLA-4 antibody administered over a two-month treatment period, over a four-month treatment period, or over a six-month or more treatment period.

In some embodiments, chemoradiation therapy comprises a platinum-based therapeutic agent.

In some embodiments, the concurrent chemoradiation therapy comprises any accepted standard first-line treatments for patients with small-cell lung cancer, squamous cell carcinoma of the head and neck and/or non-small-cell lung cancer. In some embodiments, standard first-line treatments may include chemotherapy, radiation therapy, or both (chemoradiation therapy). In some embodiments, the therapy can comprise one or more platinum-based chemotherapeutic agents. In some embodiments, the one or more platinum-based chemotherapeutic agents can be selected from carboplatin, cisplatin, oxaliplatin, or combinations thereof. As described herein, the platinum-based therapy can comprise singlet or doublet regimens such as, for example, administering cisplatin or carboplatin with another anticancer agent such as paclitaxel, docetaxel, etoposide, gemcitabine, vinorelbine, and the like.

In some embodiments provided herein is a combination comprising a human anti-PD-L1 antibody, an anti-CTLA-4 antibody, and concurrent chemoradiation therapy for use in a method of increasing the overall response rate (ORR) in a patient with SCLC.

In some embodiments provided herein is a combination comprising a human anti-PD-L1 antibody, an anti-CTLA-4 antibody, and concurrent chemoradiation therapy for use in a method increasing the disease control rate (DCR) in a patient with SCLC.

In some embodiments provided herein is a combination comprising a human anti-PD-L1 antibody, an anti-CTLA-4 antibody, and concurrent chemoradiation therapy for use in treatment of SCLC.

In some embodiments provided herein is a combination comprising a human anti-PD-L1 antibody and concurrent chemoradiation therapy for use in a method of increasing the overall response rate (ORR) in a patient with HNSCC.

In some embodiments provided herein is a combination comprising a human anti-PD-L1 antibody and concurrent chemoradiation therapy for use in a method of increasing the disease control rate (DCR) in a patient with HNSCC.

In some embodiments provided herein is a use of a combination comprising a human anti-PD-L1 antibody, an anti-CTLA-4 antibody, and concurrent chemoradiation therapy in the manufacture of a medicament for use in a method of increasing the overall response rate (ORR) in a patient with SCLC.

In some embodiments provided herein is a use of a combination comprising a human anti-PD-L1 antibody, an anti-CTLA-4 antibody, and concurrent chemoradiation therapy in the manufacture of a medicament for use in a method of increasing the disease control rate (DCR) in a patient with SCLC.

In some embodiments provided herein is a use of a combination comprising a human anti-PD-L1 antibody, an anti-CTLA-4 antibody, and concurrent chemoradiation therapy in the manufacture of a medicament for use in the treatment of SCLC.

In some embodiments provided herein is a use of a combination comprising a human anti-PD-L1 antibody and concurrent chemoradiation therapy in the manufacture of a medicament for use in a method of increasing the overall response rate (ORR) in a patient with HNSCC.

In some embodiments provided herein is a use of a combination comprising a human anti-PD-L1 antibody and concurrent chemoradiation therapy in the manufacture of a medicament for use in a method of increasing the disease control rate (DCR) in a patient with HNSCC.

In some embodiments provided herein is a use of a combination comprising a human anti-PD-L1 antibody and concurrent chemoradiation therapy in the manufacture of a medicament for use in the treatment of HNSCC.

The terms “pharmaceutical composition” or “therapeutic composition,” as used herein, refer to a compound or composition capable of inducing a desired therapeutic effect when properly administered to a subject. In some embodiments, the disclosure provides a pharmaceutical composition comprising a pharmaceutically acceptable carrier and a therapeutically effective amount of at least one antibody of the disclosure.

The terms “pharmaceutically acceptable carrier” or “physiologically acceptable carrier,” as used herein, refer to one or more formulation materials suitable for accomplishing or enhancing the delivery of one or more antibodies of the disclosure.

When used for in vivo administration, the formulations of the disclosure should be sterile. The formulations of the disclosure may be sterilized by various sterilization methods, including, for example, sterile filtration or radiation. In one embodiment, the formulation is filter sterilized with a presterilized 0.22-micron filter. Sterile compositions for injection can be formulated according to conventional pharmaceutical practice as described in “Remington: The Science & Practice of Pharmacy,” 21st ed., Lippincott Williams & Wilkins, (2005).

In some embodiments, antibodies can be formulated for particular routes of administration, such as oral, nasal, pulmonary, topical (including buccal and sublingual), rectal, vaginal, and/or parenteral administration. The terms “parenteral administration” and “administered parenterally,” as used herein, refer to modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection, and infusion. Formulations of the disclosure that are suitable for topical or transdermal administration include powders, sprays, ointments, pastes, creams, lotions, gels, solutions, patches, and inhalants. The antibodies and other actives may be mixed under sterile conditions with a pharmaceutically acceptable carrier, and with any preservatives, buffers, or propellants which may be required (see, e.g., U.S. Pat. Nos. 7,378,110; 7,258,873; and 7,135,180; U.S. Patent Application Publication Nos. 2004/0042972 and 2004/0042971).

The formulations can be presented in unit dosage form and can be prepared by any method known in the art of pharmacy. Actual dosage levels of the active ingredients in the formulation of the present disclosure may be varied so as to obtain an amount of the active ingredient which is effective to achieve the desired therapeutic response for a particular subject, composition, and mode of administration, without being toxic to the subject (e.g., “a therapeutically effective amount”). Dosages can also be administered via continuous infusion (such as through a pump). The administered dose may also depend on the route of administration. For example, subcutaneous administration may require a higher dosage than intravenous administration.

The disclosed methods of treatment can provide for substantial improvement in a patient's overall response rate (ORR), disease control rate (DCR) progression-free survival (PFS), overall survival (OS), and proportion of patients alive at 12 months from randomization (OS12).

In some embodiments, the method provides an increase in PFS relative to placebo. In some embodiments, the method provides an increase in ORR relative to placebo. In some embodiments, the method provides an increase in DCR relative to placebo. In some embodiments, the method provides an increase in OS versus placebo.

The practice of the methods disclosed herein employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology”; “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction” (Mullis, 1994); and “Current Protocols in Immunology” (Coligan, 1991).

### EXAMPLES

### Example 1: Efficacy of Durvalumab and Tremelimumab in Combination with Chemoradiation Therapy in Patients with Solid Tumors

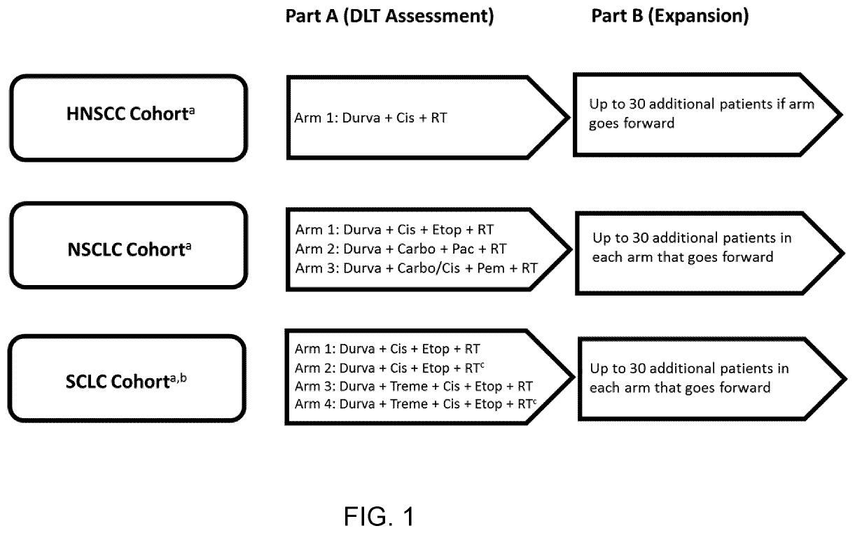

An open-label, multicenter, Phase I study was conducted to evaluate the safety, tolerability, pharmacokinetics (PK), immunogenicity, and preliminary efficacy of durvalumab tremelimumab in combination with chemoradiation in patients with advanced solid tumors. The initial cohorts included patients with locally advanced HNSCC, locally advanced, unresectable (Stage III) NSCLC, and limited-stage SCLC. The study was composed of a dose-limiting toxicity (DLT) assessment phase (Part A) and an expansion phase (Part B). A study schematic is shown in FIG. 1.

### Study Design

### HNSCC Cohort

### Part A: DLT Assessment Phase

The study included 1 arm. Details of the initial arm is as follows:
Arm 1: cisplatin with radiation+durvalumab in patients with locally advanced HNSCC

Arm 1 employed standard durvalumab doses/dosing regimens (durvalumab 1500 mg every 4 weeks [q4w]) with chemoradiation.

Over ˜7 weeks, the primary tumor and involved lymph nodes ideally received 70 Gy in 35 daily fractions (i.e,, 2 Gy/fraction, 5 fractions per week) and subclinical disease sites ideally received 56 Gy in 35 daily fractions (i.e., 1.6 Gy/fraction, 5 fractions per week). If regions considered at high risk for microscopic disease were identified, these may have been treated to a total dose of 61.25 Gy in 35 fractions (i.e., 1.75 Gy/fraction, 5 fractions per week).

In Part A, up to 12 patients in total were assigned to Arm 1 for evaluation of DLTs in patients treated with durvalumab with chemoradiation.

### Part B: Expansion Phase

In Part B, up to 30 additional patients were assigned into the arm.

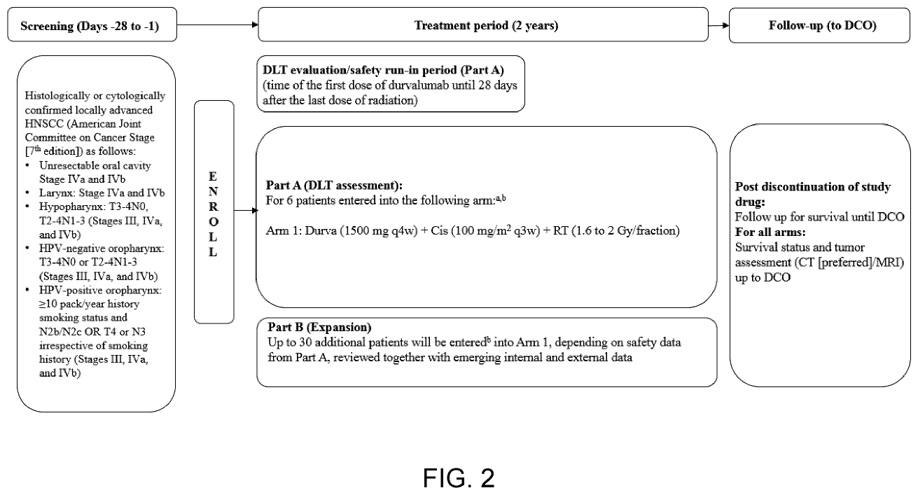

A detailed study flowchart for the HNSCC cohort is presented in FIG. 2.

### NSCLC Cohort

### Part A: DLT Assessment Phase

The study initially included 3 arms. Details of the initial arms are as follows:
Arm 1: cisplatin and etoposide with radiation+durvalumab in patients with locally advanced, unresectable (Stage III) NSCLC.
Arm 2: carboplatin and paclitaxel with radiation+durvalumab in patients with locally advanced, unresectable (Stage III) NSCLC
Arm 3 (non-squamous indication only): investigator's choice of carboplatin and pemetrexed or cisplatin and pemetrexed with radiation+durvalumab in patients with locally advanced, unresectable (Stage III) NSCLC

Arms 1, 2, and 3 employed standard durvalumab doses/dosing regimens (durvalumab 1500 mg q4w) with chemoradiation.

Over a period of ˜6 weeks, a total dose of 60 Gy was delivered in 30 daily fractions (i.e., 2 Gy/fraction, 5 fractions per week). In Part A, 18 patients in total were entered into Arms 1, 2, and 3 (6 patients per arm) for evaluation of DLTs in patients treated with durvalumab with chemoradiation.

### Part B: Expansion Phase

In Part B, up to 30 additional patients were assigned into each arm. However, Arm 3 will enroll patients with non-squamous NSCLC only.

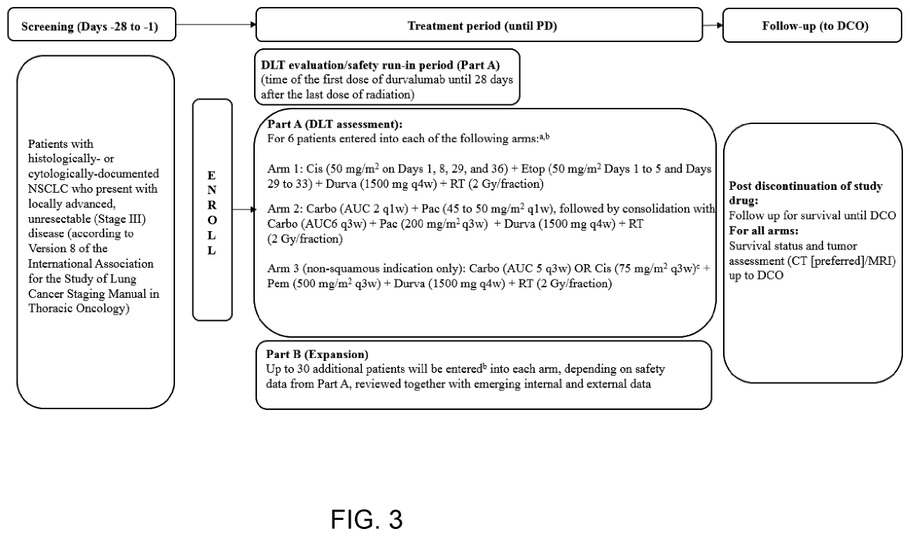

A detailed study flowchart for the NSCLC cohort is presented in FIG. 3.

### SCLC Cohort

### Part A: DLT Assessment Phase

The study included 4 arms. Details of the initial arms are as follows:
Arm 1: cisplatin and etoposide with standard radiation+durvalumab in patients with limited-stage SCLC. Patients started with cisplatin, but if cisplatin was not tolerated, they had the option to switch to carboplatin (AUC 5).
Arm 2: cisplatin and etoposide with hyperfractionated radiation+durvalumab in patients with limited-stage SCLC. Patients started with cisplatin, but if cisplatin was not tolerated, they had the option to switch to carboplatin (AUC 5).
Arm 3: cisplatin and etoposide with standard radiation+durvalumab and tremelimumab in patients with limited-stage SCLC. Patients started with cisplatin, but if cisplatin was not tolerated, they had the option to switch to carboplatin (AUC 5).
Arm 4: cisplatin and etoposide with hyperfractionated radiation+durvalumab and tremelimumab in patients with limited-stage SCLC. Patients started with cisplatin, but if cisplatin was not tolerated, they had the option to switch to carboplatin (AUC 5).

Arms 1, 2, 3, and 4 employed standard durvalumab±tremelimumab doses/dosing regimens (durvalumab 1500 mg q4w±tremelimumab 75 mg q4w) with chemoradiation administered in accordance with the National Comprehensive Cancer Network guidelines for radiotherapy and local prescribing information for chemotherapy.

Over a period of 6 to 7 weeks (starting within the first or second cycle of chemotherapy), a total dose of 60 to 70 Gy was delivered in 30 to 35 fractions (i.e., 2 Gy/fraction, 5 fractions per week) for Arms 1 and 3. Over a period of 3 weeks (starting within the first or second cycle of chemotherapy), a total dose of 45 Gy was delivered in 30 fractions (i.e., 1.5 Gy/fractions, 2 fractions per day [at least 6 hours apart], 10 fractions per week) for Arms 2 and 4.

If the starting dosing schedule (Schedule 0) of tremelimumab (75 mg q4w) in Arms 3 and 4 was not tolerated, then it was adjusted (Schedule −1); 1 dose of tremelimumab was given on Day 1 (Week 0) and 3 further doses post-chemoradiation. If this schedule was still not tolerated, then the tremelimumab schedule was adjusted once more (Schedule −2), wherein all 4 doses will be given post-chemoradiation (Table 1). For Schedule −1 and Schedule −2, post-chemoradiation tremelimumab dosing commenced once initial chemoradiation had been completed and at least 48 hours after any recommended prophylactic cranial irradiation (PCI) and was administered alongside the next scheduled dose of durvalumab.

TABLE 1
Tremelimumab schedule intensity changes (for tremelimumab-
containing arms) in descending order

| Tremelimumab | Week | Week | Week | Week | Week | Week | Week | Week | Week |
|---|---|---|---|---|---|---|---|---|---|
| schedule | 0 | 4 | 8 | 12 | 16 | 20 | 24 | 28 | 32 |

| | | | | | |
|---|---|---|---|---|---|
| Schedule 0 | X | X | X | X | PCI may be given after completion of |
| 75 mg | | | | | chemoradiation and at least 2 weeks after |
| tremelimumab | | | | | last dose of tremelimumab^{a} |
| Schedule −1^{c} | X | | | | PCI may be given after completion of |
| 75 mg | | | | | chemoradiation, followed by 3 post- |
| tremelimumab | | | | | chemoradiation/PCI doses of |
| | | | | | tremelimumab^{a, b} |
| Schedule −2 | | | | | PCI may be given after completion of |
| 75 mg | | | | | chemoradiation, followed by 4 post- |
| tremelimumab | | | | | chemoradiation/PCI doses of |
| | | | | | tremelimumab^{a, b} |

^{a}PCI is permitted per the investigator's discretion for patients who respond to initial therapy (CR or PR). PCI should be administered in accordance with the Error! Reference source not found. guidelines. Where indicated, PCI will be given after resolution of acute toxicity initial chemoradiation therapy and no earlier than 2 weeks after last dose of tremelimumab.
^{b}Maintenance doses of tremelimumab should be administered q4w alongside the next scheduled dose of durvalumab and at least 48 hours after completion of PCI in eligible patients.
^{c}Commencement of thoracic radiation in subsequent patients can be limited to within second cycle of chemotherapy, at the discretion of the safety review committee, if DLTs occur in the first cycle of chemoradiotherapy in schedule 0).
CR Complete response; PCI Prophylactic cranial irradiation; PR Partial response; q4w Every 4 weeks.

PCI was permitted per the investigator's discretion for patients in the SCLC cohort who respond to initial therapy (complete response [CR] or partial response [PR]). Where indicated, PCI was given after resolution of acute toxicities of initial chemoradiation therapy and no earlier than 2 weeks after last dose of tremelimumab. It was recommended to give PCI to eligible patients 4 to 11 weeks after completion of chemoradiation. Patients in Arms 1 and 2 received PCI after resolution of acute toxicities from initial chemoradiation therapy. For patients in Arms 3 and 4, those on Schedule 0 received PCI after resolution of acute toxicities from initial chemoradiation therapy, and at least 2 weeks after the last dose of tremelimumab, while patients on Schedule −1 and Schedule −2 received PCI after resolution of acute toxicities of initial chemoradiation therapy and before commencing the post-chemoradiation doses of tremelimumab. In Part A, 12 patients in total were entered into Arms 1 and 2 for evaluation of DLTs in patients treated with durvalumab with chemoradiation. An additional 6 patients were entered into each arm if additional safety data was needed.

### Part B: Expansion Phase

In the expansion phase, the investigator could choose either cisplatin or carboplatin, in combination with etoposide, to further evaluate the safety and tolerability of concurrent chemoradiation with durvalumab and/or tremelimumab. A minimum of 5 patients was assigned to the carboplatin regimen in each arm that goes forward. Up to 30 additional patients were entered into each arm that goes forward.

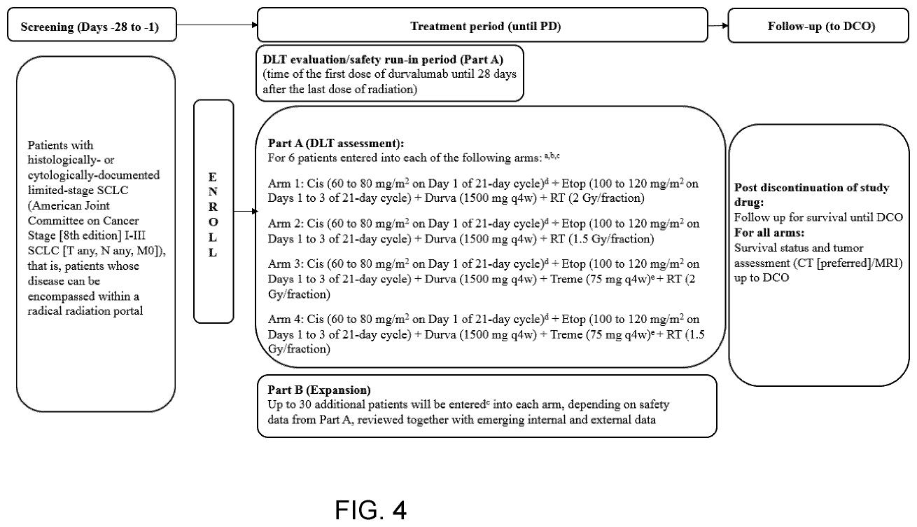

A detailed study flowchart for the SCLC cohort is presented in FIG. 4.

### Objectives

Primary objective

| Primary objective: | Outcome measures: |
|---|---|
| To assess the safety and tolerability profile of | DLTs |
| durvalumab ± tremelimumab in combination | Descriptive measures of AEs, physical examinations, |
| with chemoradiation. | laboratory findings (including clinical chemistry, |
| | hematology, and urinalysis), vital signs (including BP |
| | and pulse), ECGs |

Secondary objective

| Secondary objective: | Outcome measures: |
|---|---|
| To assess the efficacy/antitumor activity of | ORR, BoR, DoR, and DCR using |
| durvalumab ± tremelimumab in combination | investigator's assessments according to |
| with chemoradiation | RECIST 1.1 (all cohorts), and OS (all |
| | cohorts) |
| | DFS, DFS12, DFS18, and DFS24 using |
| | investigator's assessments according to |
| | RECIST 1.1 (HNSCC cohort) |
| | PFS12, PFS18, and PFS24 using |
| | investigator's assessments according to |
| | RECIST 1.1 (NSCLC and SCLC cohorts) |

AE Adverse event; BP Blood pressure; DLT Dose-limiting toxicity; ECG Electrocardiogram.
BoR Best objective response; DCR Disease control rate; DFS Disease-free survival; DFS12 Disease-free survival at 12 months; DFS18 Disease-free survival at 18 months; DFS24 Disease-free survival at 24 months; DoR Duration of response; HNSCC Squamous cell carcinoma of the head and neck; NSCLC Non-small-cell lung cancer; ORR Objective response rate; OS Overall survival; PFS12 Progression-free survival at 12 months; PFS18 Progression-free survival at 18 months; PFS24 Progression-free survival at 24 months; RECIST 1.1 Response Evaluation Criteria in Solid Tumors, Version 1.1; SCLC Small-cell lung cancer.

### Target Patient Population

For inclusion in the study, patients fulfilled the following criteria: age ≥18 years at the time of screening and no prior exposure to immune-mediated therapy, including, but not limited to, other anti-CTLA-4, anti-PD-1, anti-PD-L1, anti-PD-L2 antibodies, or therapeutic anticancer vaccines.

For the HNSCC cohort, patients had histologically or cytologically confirmed locally advanced HNSCC who were eligible for definitive chemoradiation treatment and not considered for primary surgery based on investigator decision or patient refusal. Patients with the following sub-types of HNSCC were eligible for this study:
Unresectable oral cavity Stage IVa and IVb
Larynx: Stage IVa and IVb
Hypopharynx: T3-4N0, T2-4N1-3 (Stage III, IVa, and IVb)
HPV-negative oropharynx: T3-4N0 or T2-4N1-3 (Stage III, IVa, and IVb)
HPV-positive oropharynx: ≥10 pack/year history smoking status and N2b/N2c or T4 or N3 irrespective of smoking history (Stages III, IVa, and IVb)

For the HNSCC cohort, NSCLC cohort, patients had histologically or cytologically documented NSCLC who present with locally advanced, unresectable (Stage III) disease.

For the SCLC cohort, patients had histologically or cytologically documented limited-stage SCLC (I-III SCLC [T any, N any, MO]), that is, patients whose disease can be encompassed within a radical radiation portal.

### Efficacy Assessments

Efficacy assessments of ORR, best objective response (BoR), DoR, DCR, and PFS were derived using Investigator RECIST 1.1 assessments. Investigator RECIST 1.1 tumor assessments at baseline utilized images from CT (preferred) or MRI, each preferably with IV contrast, of the applicable region(s) for the tumor type of that cohort. FDG-PET/CT and brain MRI with IV contrast (preferred; or CT with IV contrast) scans were acquired during the screening period for staging purposes (to identify metastatic tumor sites) and as comparators for any identical on-study scans to assess new lesions, and they were not to be used for baseline RECIST 1.1 selection of target lesions (TLs) and non-target lesions (NTLs). Any other areas of disease involvement were additionally imaged based on the signs and symptoms of individual patients. The baseline assessment was performed no more than 28 days before start of study drug and ideally was performed as close as possible to and prior to the start of study drug. The RECIST 1.1 assessments of baseline images identified up to 5 TLs total, no more than 2 TLs per organ (defined measurable) and NTLs, and each lesion (and any new lesion) was evaluated in subsequent follow-up images. This allowed determination of follow-up TL response, NTL response, new lesions, and overall timepoint tumor responses (complete response [CR], partial response [PR], stable disease [SD], progressive disease [PD], or not evaluable [NE]).

### Dosage, and Mode of Administration

For all cohorts, where applicable, if a patient's weight fell to 30 kg or below (≤30 kg), then the patient received weight-based dosing equivalent to 20 mg/kg of durvalumab q4w and 1 mg/kg tremelimumab q4w until the weight improved to more than 30 kg (>30 kg), at which point the patient started receiving the fixed dosing of durvalumab 1500 mg plus tremelimumab 75 mg q4w.

On days when multiple intraperitoneals (IPs) were administered, IV tremelimumab was administered first, followed by durvalumab and then cisplatin, as applicable.

Tremelimumab was administered first; the durvalumab infusion started immediately after the end of the tremelimumab infusion. Standard infusion time for both tremelimumab and durvalumab was 1 hour (±15 minutes); however, if there were interruptions during infusion, the total allowed time did exceed 8 hours at room temperature. If there were clinical concerns related to tolerability of immediate sequential administration, then at the discretion of the investigator, subsequent cycles of durvalumab were given up to 1 hour after the end of tremelimumab infusion.

If durvalumab and chemotherapy were administered on the same day, durvalumab was administered first, followed by chemotherapy (infusion times per institutional standards).

Radiation was administered according to the recommended treatment guidance in the National Comprehensive Cancer Network.

### HNSCC Cohort

Durvalumab (MEDI4736) Monotherapy with Chemoradiation (Arm 1)

Patients in Arm 1 received the following:

Durvalumab: patients received 1500 mg durvalumab (MEDI4736) via IV infusion q4w for 2 years, unless there was unacceptable toxicity, withdrawal of consent, or another discontinuation criterion was met (whichever occurred first).

Radiation: external beam radiation (1.6 to 2 Gy/fraction) was administered daily beginning at Week 1 through Week 7 (5 fractions per week over ˜7 weeks of treatment), unless there was unacceptable toxicity, withdrawal of consent, or another discontinuation criterion was met (whichever occurred first).

Cisplatin: patients received cisplatin 100 mg/m² via IV infusion q3w beginning Week 1, with the last dose at Week 7 (3 doses), unless there was unacceptable toxicity, withdrawal of consent, or another discontinuation criterion was met (whichever occurs first). Table 2 depicts the dosing scheme for the HNSCC cohort.

TABLE 2
Dosing scheme for HNSCC cohort

| | | | | | | | | | | Post- | |
|---|---|---|---|---|---|---|---|---|---|---|---|
| | During chemoradiation | | | | | | | | | chemoradiation | |
| | Cycle | | | | | | | | | | |
| | 1 | | | | 2 | | | | 3 | 4 | 5 to 14 |
| | Week (±3 days) | | | | | | | | | | |
| Treatment | | | | | | | | | | | 16 to 2 |
| arms | 0 | 1 | 2 | 3 | 4 | 5 | 6 | 7 | 8 | 12 | years^{a} |

| | | | | | | | |
|---|---|---|---|---|---|---|---|
| Durva + | Durva | Cis | Durva + | Cis | Durva | Durva | Durva |
| chemoradiation | | | Cis | | | | |

| | |
|---|---|
| (Arm 1) | Radiation (daily administration per the National |
| | Comprehensive Cancer Network guidelines) |

^{a}Note that the last dose of durvalumab is administered at Week 100.
Note:
All drugs administered have a dosing window of ±3 days (except Day 1, which will have a dosing window of +3 days).
Note:
Durvalumab dose will be 1500 mg; cisplatin dose will be 100 mg/m²; radiation will be 1.6 to 2 Gy/fraction, 5 fractions per week.
Cis Cisplatin; Durva Durvalumab; HNSCC Squamous cell carcinoma of the head and neck.

### NSCLC Cohort

Durvalumab (MEDI4736) Monotherapy with Chemoradiation (Arm 1)

Patients in Arm 1 received the following:

Durvalumab: patients received 1500 mg durvalumab (MEDI4736) via IV infusion q4w up to PD, unless there was unacceptable toxicity, withdrawal of consent, or another discontinuation criterion was met (whichever occurred first).

Radiation: external beam radiation (2 Gy/fraction) was administered daily beginning at Week 0 through Week 5 (5 fractions per week over ˜6 weeks of treatment, for a total of 60 Gy), unless there was unacceptable toxicity, withdrawal of consent, or another discontinuation criterion was met (whichever occurred first).

Cisplatin: patients received cisplatin 50 mg/m² via IV infusion on Days 1, 8, 29, and 36 (Weeks 0, 1, 4, and 5, respectively), unless there was unacceptable toxicity, withdrawal of consent, or another discontinuation criterion was met (whichever occurred first).

Etoposide: patients received etoposide 50 mg/m² via IV infusion on Days 1 to 5 (Week 0) and Days 29 to 33 (Week 4), unless there was unacceptable toxicity, withdrawal of consent, or another discontinuation criterion was met (whichever occurred first).

Durvalumab (MEDI4736) Monotherapy with Chemoradiation (Arm 2)

Patients in Arm 2 received the following:

Durvalumab: patients received 1500 mg durvalumab (MEDI4736) via IV infusion q4w up to PD, unless there was unacceptable toxicity, withdrawal of consent, or another discontinuation criterion was met (whichever occurred first).

Radiation: external beam radiation (2 Gy/fraction) was administered daily beginning at Week 0 through Week 5 (5 fractions per week over ˜6 weeks of treatment, for a total of 60 Gy) unless there was unacceptable toxicity, withdrawal of consent, or another discontinuation criterion is met (whichever occurred first).

Carboplatin: patients received carboplatin AUC 2 via IV infusion q1w beginning Week 0 with the last dose at Week 5 (6 doses), followed by consolidation with carboplatin AUC 6 via IV infusion on Day 43 (Week 6) and Day 64 (Week 9) (Day 1 of a 21-day cycle [q3w] for 2 cycles), unless there was unacceptable toxicity, withdrawal of consent, or another discontinuation criterion was met (whichever occurred first). Consolidation cycles of carboplatin were optional and dependent on local practice.

Paclitaxel: patients received paclitaxel 45 to 50 mg/m2 via IV infusion q1w beginning Week 0 with the last dose at Week 5 (6 doses), followed by consolidation with paclitaxel 200 mg/m2 via IV infusion on Day 43 (Week 6) and Day 64 (Week 9) (Day 1 of a 21-day cycle [q3w] for 2 cycles), unless there was unacceptable toxicity, withdrawal of consent, or another discontinuation criterion was met (whichever occurred first). Consolidation cycles of paclitaxel were optional and dependent on local practice.

Durvalumab (MEDI4736) Monotherapy with Chemoradiation (Arm 3)

Patients in Arm 3 received the following:

Durvalumab: patients received 1500 mg durvalumab (MEDI4736) via IV infusion q4w up to PD, unless there was unacceptable toxicity, withdrawal of consent, or another discontinuation criterion was met (whichever occurs first).

Radiation: external beam radiation (2 Gy/fraction) was administered daily beginning at Week 0 through Week 5 (5 fractions per week over ˜6 weeks of treatment, for a total of 60 Gy), unless there was unacceptable toxicity, withdrawal of consent, or another discontinuation criterion was met (whichever occurred first).

Carboplatin/cisplatin: at the discretion of the investigator, patients received carboplatin AUC 5 or cisplatin 75 mg/m² via IV infusion on Day 1 (Week 0), Day 22 (Week 3), and Day 43 (Week 6) (Day 1 of a 21-day cycle [q3w] for 3 cycles), unless there was unacceptable toxicity, withdrawal of consent, or another discontinuation criterion was met (whichever occurred first).

Pemetrexed: patients received pemetrexed 500 mg/m² via IV infusion on Day 1 (Week 0), Day 22 (Week 3), and Day 43 (Week 6) (Day 1 of a 21-day cycle [q3w] for 3 cycles), unless there was unacceptable toxicity, withdrawal of consent, or another discontinuation criterion was met (whichever occurred first).

Table 3 depicts the dosing scheme for the NSCLC cohort.

TABLE 3
Dosing scheme for NSCLC cohort

| | | | | | | | | | | | Post- |
|---|---|---|---|---|---|---|---|---|---|---|---|
| | During chemoradiation | | | | | | | | | | chemoradiation |
| | Cycle | | | | | | | | | | |
| | 1 | | | | 2 | | | | 3 | | 4 to PD |
| Treatment | Week (±3 days) | | | | | | | | | | |
| arms | 0 | 1 | 2 | 3 | 4 | 5 | 6 | 7 | 8 | 9 | 12 to PD |
| Arm 1 | Durva + | Cisa | | | Durva + | Cis^{a} | | | Durva | | Durva |
| | Cis^{a} + | | | | Cis^{a} + | | | | | | |
| | Etop^{b} | | | | Etop^{b} | | | | | | |
| | Radiation (daily administration per the National Comprehensive | | | | | | | | | | |
| | Cancer Network guidelines) | | | | | | | | | | |
| Arm 2 | Durva + | Carbo + | Carbo + | Carbo + | Durva + | Carbo + | Carbo + | | Durva | Carbo + | Durva |
| | Carbo + | Pac^{c} | Pac^{c} | Pac^{c} | Carbo + | Pac^{c} | Pac^{d} | | | Pac^{d} | |
| | Pac^{c} | | | | Pac^{c} | | | | | | |
| | Radiation (daily administration per the National Comprehensive | | | | | | | | | | |
| | Cancer Network guidelines) | | | | | | | | | | |
| Arm 3 | Durva + | | | Carbo or | Durva | | Carbo or | | Durva | | Durva |
| | Carbo or | | | Cis^{e} + | | | Cis^{e} + | | | | |
| | Cis^{e} + | | | Pem^{f} | | | Pem^{f} | | | | |
| | Pem^{f} | | | | | | | | | | |

Radiation (daily administration per the National Comprehensive
Cancer Network guidelines)

^{a}Cisplatin 50 mg/m² will be administered to patients in Arm 1 on Days 1, 8, 29, and 36.
^{b}Etoposide 50 mg/m² will be administered to patients in Arm 1 on Days 1 to 5 and Days 29 to 33.
^{c}Carboplatin AUC 2 and paclitaxel (45 to 50 mg/m²) will be administered to patients in Arm 2 q1w from Weeks 0 to 5.
^{d}Consolidation with carboplatin AUC 6 and paclitaxel (200 mg/m²) will be administered to patients in Arm 2 on Day 43 and Day 64 (Day 1 of a 21-day cycle [q3w] for 2 cycles). Consolidation cycles of carboplatin/paclitaxel are optional and dependent on local practice.
^{e}For patients in Arm 3, the investigator may decide whether to administer carboplatin AUC 5 or cisplatin (75 mg/m²) on Day 1, Day 22, and Day 43 (Day 1 of a 21-day cycle [q3w] for 3 cycles).
^{f}Pemetrexed (500 mg/m²) will be administered on Day 1, Day 22, and Day 43 (Day 1 of a 21-day cycle [q3w] for 3 cycles) to patients in Arm 3.
Note:
All drugs administered have a dosing window of ±3 days (except Day 1, which will have a dosing window of +3 days).
Note:
Durvalumab dose will be 1500 mg (q4w) and radiation will be 2 Gy/fraction, 5 fractions per week (for a total of 60 Gy).
AUC Area under the plasma drug concentration-time curve;
Carbo Carboplatin;
Cis Cisplatin;
Durva Durvalumab;
Etop Etoposide;
NSCLC Non-small-cell lung cancer;
Pac Paclitaxel;
Pem Pemetrexed:
PD Progressive disease;
q1w Once weekly;
q3w Every 3 weeks;
q4w Every 4 weeks

### SCLC Cohort

Durvalumab (MEDI4736) Monotherapy with Chemoradiation (Arm 1)

Patients in Arm 1 received the following:

Durvalumab: patients received 1500 mg durvalumab (MEDI4736) via IV infusion q4w up to PD, unless there was unacceptable toxicity, withdrawal of consent, or another discontinuation criterion was met (whichever occurred first).

Radiation: external beam radiation (2 Gy/fraction) was administered daily, starting within the first or second cycle of chemotherapy; 5 fractions per week over 6 to 7 weeks of treatment for a total of 60 to 70 Gy, unless there was unacceptable toxicity, withdrawal of consent, or another discontinuation criterion was met (whichever occurred first).

Cisplatin and etoposide: patients received cisplatin 60 to 80 mg/m² via IV infusion on Day 1 (Week 0), Day 22 (Week 3), Day 43 (Week 6), Day 64 (Week 9), Day 85 (Week 12), and Day 106 (Week 15) (Day 1 of a 21-day cycle [q3w] for 4 to 6 cycles, as per local guidelines) and etoposide 100 to 120 mg/m² on Days 1 to 3 (Week 0), Days 22 to 24 (Week 3), Days 43 to 45 (Week 6), Days 64 to 66 (Week 9), Days 85 to 87 (Week 12), and Days 106 to 108 (Week 15) (Days 1 to 3 of a 21-day cycle for 4 to 6 cycles, as per local guidelines), starting on Week 0, unless there was unacceptable toxicity, withdrawal of consent, or another discontinuation criterion is met (whichever occurred first). Patients started with cisplatin, but if cisplatin was not tolerated, they had the option to switch to carboplatin (AUC 5). The total cisplatin dose per cycle may be split over Days 1, 2, and 3 of each chemotherapy cycle in Part B only, if there was tolerability concerns and only after consultation with the Global Medical Team.

Durvalumab (MEDI4736) Monotherapy with Chemoradiation (Arm 2)

Patients in Arm 2 received the following:

Durvalumab: patients received 1500 mg durvalumab (MEDI4736) via IV infusion q4w up to PD, unless there wa unacceptable toxicity, withdrawal of consent, or another discontinuation criterion was met (whichever occurred first).

Radiation: external beam radiation (1.5 Gy/fraction) was administered daily, starting within the first or second cycle of chemotherapy; 10 fractions per week [2 fractions per day, at least 6 hours apart] over 3 weeks of treatment for a total of 45 Gy, unless there was unacceptable toxicity, withdrawal of consent, or another discontinuation criterion was met (whichever occurred first).

Cisplatin and etoposide: patients received cisplatin 60 to 80 mg/m² via IV infusion on Day 1 (Week 0), Day 22 (Week 3), Day 43 (Week 6), Day 64 (Week 9), Day 85 (Week 12), and Day 106 (Week 15) (Day 1 of a 21-day cycle [q3w] for 4 to 6 cycles, as per local guidelines) and etoposide 100 to 120 mg/m² on Days 1 to 3 (Week 0), Days 22 to 24 (Week 3), Days 43 to 45 (Week 6), Days 64 to 66 (Week 9), Days 85 to 87 (Week 12), and Days 106 to 108 (Week 15) (Days 1 to 3 of a 21-day cycle for 4 to 6 cycles, as per local guidelines), starting on Week 0, unless there was unacceptable toxicity, withdrawal of consent, or another discontinuation criterion was met (whichever occurred first). Patients started with cisplatin, but if cisplatin was not tolerated, they had the option to switch to carboplatin (AUC 5). The total cisplatin dose per cycle may be split over Days 1, 2, and 3 of each chemotherapy cycle in Part B only, if there are tolerability concerns and only after consultation with the Global Medical Team.

Durvalumab (MEDI4736)+Tremelimumab Combination Therapy with Chemoradiation (Arm 3)

Patients in Arm 3 received the following:

Durvalumab and tremelimumab: patients received durvalumab (MEDI4736) (1500 mg q4w) in combination with tremelimumab (75 mg IV q4w; 4 doses in total), followed by durvalumab (MEDI4736) 1500 mg q4w up to PD, unless there was unacceptable toxicity, withdrawal of consent, or another discontinuation criterion was met (whichever occurred first).

If the starting dosing schedule (Schedule 0) of tremelimumab (75 mg q4w) in Arm 3 was not tolerated, then it was adjusted (Schedule −1); 1 dose of tremelimumab was given on Day 1 (Week 0) and 3 further doses q4w post-chemoradiation. If this schedule was still not tolerated, then the tremelimumab schedule was adjusted once more (Schedule −2), wherein all doses will be given post-chemoradiation (Table 1). For Schedule −1 and Schedule −2, tremelimumab maintenance dosing commenced once initial chemoradiation was completed and at least 48 hours after any recommended PCI and administered alongside the next scheduled dose of durvalumab.

Radiation: external beam radiation (2 Gy/fraction) was administered daily, starting within the first or second cycle of chemotherapy; 5 fractions per week over 6 to 7 weeks of treatment for a total of 60 to 70 Gy, unless there was unacceptable toxicity, withdrawal of consent, or another discontinuation criterion was met (whichever occurred first).

Cisplatin and etoposide: patients received cisplatin 60 to 80 mg/m² via IV infusion on Day 1 (Week 0), Day 22 (Week 3), Day 43 (Week 6), Day 64 (Week 9), Day 85 (Week 12), and Day 106 (Week 15) (Day 1 of a 21-day cycle [q3w] for 4 to 6 cycles, as per local guidelines) and etoposide 100 to 120 mg/m² on Days 1 to 3 (Week 0), Days 22 to 24 (Week 3), Days 43 to 45 (Week 6), Days 64 to 66 (Week 9), Days 85 to 87 (Week 12), and Days 106 to 108 (Week 15) (Days 1 to 3 of a 21-day cycle for 4 to 6 cycles, as per local guidelines), starting on Week 0, unless there was unacceptable toxicity, withdrawal of consent, or another discontinuation criterion was met (whichever occurred first). Patients started with cisplatin, but if cisplatin was not tolerated, they had the option to switch to carboplatin (AUC 5). The total cisplatin dose per cycle may be split over Days 1, 2, and 3 of each chemotherapy cycle in Part B only, if there are tolerability concerns and only after consultation with the Global Medical Team.

Durvalumab (MEDI4736)+Tremelimumab Combination Therapy with Chemoradiation (Arm 4)

Patients in Arm 4 will receive the following:

Durvalumab and tremelimumab: patients received durvalumab (MEDI4736) (1500 mg q4w) in combination with tremelimumab (75 mg IV q4w; 4 doses in total), followed by durvalumab (MEDI4736) 1500 mg q4w up to PD, unless there was unacceptable toxicity, withdrawal of consent, or another discontinuation criterion was met (whichever occurred first).

If the starting dosing schedule (Schedule 0) of tremelimumab (75 mg q4w) in Arm 4 was not tolerated, then it was adjusted (Schedule −1); 1 dose of tremelimumab was given on Day 1 (Week 0) and 3 further doses q4w post-chemoradiation. If this schedule was still not tolerated, then the tremelimumab schedule was adjusted once more (Schedule −2), wherein all doses will be given post-chemoradiation (Table 1). For Schedule −1 and Schedule −2, tremelimumab maintenance dosing commenced once initial chemoradiation was completed and at least 48 hours after any recommended PCI and was administered alongside the next scheduled dose of durvalumab.

Radiation: external beam radiation (1.5 Gy/fraction) was administered daily, starting within the first or second cycle of chemotherapy; 10 fractions per week; 2 fractions per day, at least 6 hours apart; over 3 weeks of treatment for a total of 45 Gy, unless there was unacceptable toxicity, withdrawal of consent, or another discontinuation criterion was met (whichever occurred first).

Cisplatin and etoposide: patients received cisplatin 60 to 80 mg/m² via IV infusion on Day 1 (Week 0), Day 22 (Week 3), Day 43 (Week 6), Day 64 (Week 9), Day 85 (Week 12), and Day 106 (Week 15) (Day 1 of a 21-day cycle [q3w] for 4 to 6 cycles, as per local guidelines) and etoposide 100 to 120 mg/m² on Days 1 to 3 (Week 0), Days 22 to 24 (Week 3), Days 43 to 45 (Week 6), Days 64 to 66 (Week 9), Days 85 to 87 (Week 12), and Days 106 to 108 (Week 15) (Days 1 to 3 of a 21-day cycle for 4 to 6 cycles, as per local guidelines), starting on Week 0, unless there was unacceptable toxicity, withdrawal of consent, or another discontinuation criterion was met (whichever occurred first). Patients started with cisplatin, but if cisplatin was not tolerated, they had the option to switch to carboplatin (AUC 5). The total cisplatin dose per cycle may be split over Days 1, 2, and 3 of each chemotherapy cycle in Part B only, if there are tolerability concerns and only after consultation with the Global Medical Team.

Table 4 depicts the dosing scheme for the SCLC cohort.

TABLE 4
Dosing scheme for SCLC cohort

| | During chemoradiation | | | | | | | | | | Post-chemoradiation | | | | | |
|---|---|---|---|---|---|---|---|---|---|---|---|---|---|---|---|---|
| | Cycle | | | | | | | | | | | | | | | |
| | 1 | | | | 2 | | | | 3 | | 4 | | 5 | 6 | 7 | 8 to PD |
| Treatment | Week (±3 days) | | | | | | | | | | | | | | | |
| arms | 0 | 1 | 2 | 3 | 4 | 5 | 6 | 7 | 8 | 9 | 12 | 15 | 16 | 20 | 24 | 28 to PD |
| Durva + | Durva + | | | Cis + | Durva | | Cis + | | Durva | Cis + | Durva + | Cis^{f} + | Durva | Durva | Durva | Durva |
| Chemo- | Cis + | | | Etop | | | Etop | | | Etop | Cis^{f} + | Etop^{f} | | | | |
| radiation | Etop | | | | | | | | | | Etop | | | | | |
| (Arm | Radiation (daily administration per the National Comprehensive | | | | | | | | | | | | | | | |
| 1)^{a, b, c} | Cancer Network guidelines; starting within the first | | | | | | | | | | | | | | | |
| | or second cycle of chemotherapy for 6 to 7 weeks) | | | | | | | | | | | | | | | |
| Durva + | Durva + | | | Cis + | Durva | | Cis + | | Durva | Cis + | Durva + | Cis^{f} + | Durva | Durva | Durva | Durva |
| Chemo- | Cis + | | | Etop | | | Etop | | | Etop | Cis^{f} + | Etop^{f} | | | | |
| radiation | Etop | | | | | | | | | | Etop | | | | | |
| (Arm | Radiation (daily administration per the National | | | | | | | | | | | | | | | |
| 2)^{a, b, c} | Comprehensive Cancer Network guidelines; starting within | | | | | | | | | | | | | | | |
| | the first or second cycle of chemotherapy for 3 weeks) | | | | | | | | | | | | | | | |
| Durva + | Durva + | | | Cis + | Durva + | | Cis + | | Durva + | Cis + | Durva + | Cis^{f} + | Durva | Durva | Durva | Durva |
| Treme + | Treme + | | | Etop | Treme | | Etop | | Treme | Etop | Treme + | Etop^{f} | | | | |
| Chemo- | Cis + | | | | | | | | | | Cis^{f} + | | | | | |
| radiation | Etop | | | | | | | | | | Etop^{f} | | | | | |
| (Arm | Radiation (daily administration per the National Comprehensive | | | | | | | | | | | | | | | |
| 3)^{a, b, c, d, e} | Cancer Network guidelines; starting within the first | | | | | | | | | | | | | | | |
| | or second cycle of chemotherapy for 6 to 7 weeks) | | | | | | | | | | | | | | | |
| Durva + | Durva + | | | Cis + | Durva + | | Cis + | | Durva + | Cis + | Durva + | Cis^{f} + | Durva | Durva | Durva | Durva |
| Treme + | Treme + | | | Etop | Treme | | Etop | | Treme | Etop | Treme + | Etop^{f} | | | | |
| Chemo- | Cis + | | | | | | | | | | Cis^{f} + | | | | | |
| radiation | Etop | | | | | | | | | | Etop^{f} | | | | | |

| | |
|---|---|
| (Arm | Radiation (daily administration per the National |
| 4)^{a, b, c, d, e} | Comprehensive Cancer Network guidelines; starting within |
| | the first or second cycle of chemotherapy for 3 weeks) |

^{a}Cisplatin 60 to 80 mg/m² will be administered to patients in Arms 1, 2, 3, and 4 on Day 1, Day 22, Day 43, Day 64, Day 85, and Day 106 (Day 1 of a 21-day cycle [q3w] for 4 to 6 cycles), per local guidelines.
^{b}Etoposide 100 to 120 mg/m² will be administered to patients in Arms 1, 2, 3, and 4 on Days 1 to 3, Days 22 to 24, Days 43 to 45, Days 64 to 66, Days 85 to 87, and Days 106 to 108 (Days 1 to 3 of a 21-day cycle for 4 to 6 cycles), per local guidelines.
^{c}Patients should start with cisplatin, but if cisplatin is not tolerated, they have the option to switch to carboplatin (AUC 5). The total cisplatin dose per cycle may be split over Days 1, 2, and 3 of each chemotherapy cycle in Part B only, if there are tolerability concerns and only after consultation with the Global Medical Team.
^{d}If the starting dosing schedule (Schedule 0) of tremelimumab (75 mg q4w) in Arms 3 and 4 is not tolerated, then it will be adjusted (Schedule −1); 1 dose of tremelimumab will be given on Day 1 (Week 0) and 3 further doses q4w post-chemoradiation. If this schedule is still not tolerated, then the tremelimumab schedule will be adjusted once more (Schedule −2), wherein all doses will be given post-chemoradiation (Table 1). For Schedule −1 and Schedule −2, tremelimumab maintenance dosing can commence once initial chemoradiation has been completed and at least 48 hours after any recommended PCI and should be administered alongside the next scheduled dose of durvalumab. PCI should be administered in accordance with the National Comprehensive Cancer Network guidelines.
^{e}Arm 3 will only be opened if the regimen in Arm 1 is safe and tolerable, and Arm 4 will only be opened if the regimen in Arm 2 is safe and tolerable.
^{f}For patients receiving 5 or 6 cycles of chemotherapy.
Note:
All drugs administered have a dosing window of ±3 days (except Day 1, which will have a dosing window of +3 days).
Note:
Durvalumab dose will be 1500 mg (q4w); tremelimumab dose will be 75 mg; cisplatin dose will be 60 to 80 mg/m²; etoposide dose will be 100 to 120 mg/m²; radiation will be 2 Gy/fraction in Arms 1 and 3, 5 fractions per week (for a total of 60 to 70 Gy), and 1.5 Gy/fraction, 10 fractions per week (2 fractions per day, at least 6 hours apart, for a total of 45 Gy), in Arms 2 and 4.

### Results

### HNSCC

There were a total of 8 patients enrolled in the HNSCC cohort. Overall, the safety and tolerability profile of durvalumab+cisplatin+RT was manageable and consistent with the known safety profile of durvalumab alone and cisplatin+RT. No unexpected safety findings were observed for this combination regimen. Specifically, patients in the HNSCC cohort showed:
No DLTs;
Adverse events of CTCAE Grade 3 or 4 were reported for 7 patients, most frequently lymphopenia, stomatitis, amylase increased, and leukopenia;
Serious adverse events (SAEs) were experienced by 2 patients (subdural hemorrhage, acute kidney injury);
3 patients with AEs led to discontinuation of any study treatment; of these, 1 led to discontinuation of durvalumab (acute kidney injury—also the only fatal AE);
5 patients with immune-mediated adverse events (imAEs); most common group term category was hypothyroid and pancreatitis. No imAEs led to discontinuation or death.

The efficacy for the HNSCC cohort showed a 71% overall response rate (ORR); disease control rate (DCR) of 86% at 18 weeks and 83% at 48 weeks; disease free survival of 85.7% at 12 months, and 51.4% at 18 and 24 months; and overall survival of 20.1 months.

### NSCLC

There were a total of 64 patients enrolled in the HNSCC cohort. Overall, the safety findings were consistent with the known safety profiles of durvalumab and cCRT and there was no evidence that addition of durvalumab to standard of care chemoradiotherapy affected the tolerability of standard of care chemoradiotherapy. Specifically, patients in the NSCLC cohort showed:
1 DLT in Arm 1 (Grade 3 AST increased/Grade 4 ALT increased) leading to discontinuation of study treatment;
AE of CTC grade 3/4 and SAEs were experienced by 77% and 60% of patients; most of these events were expected based on the established toxicity profiles of chemotherapy, radiotherapy, and immunotherapy or because of the patient's primary tumor location and/or medical history;
Fatal cardiac events occurred in 4 patients. All patients had significant smoking history and 3/4 patients had significant cardiac-related MH. An additional 4 patients experienced fatal AEs (acute respiratory failure, pneumonia, PCP pneumonia, sepsis);
14 patients had AEs leading to discontinuation of any study treatment (n=13, durvalumab), which included 8 patients with fatal AEs and 1 patient with a DLT;
Oesophagitis (42%), radiation pneumonitis (15%), and pneumonitis (25%) events were primarily Grade 1/2 in severity. Events were manageable and rarely led to treatment discontinuation; no events had a fatal outcome;
20 patients with imAEs across the 3 treatment arms; 2 patients experienced imAEs that led to discontinuation (hepatic events, pneumonitis, no events had a fatal outcome).

The efficacy for the NSCLC cohort showed an ORR for each arm of 66.7%, 54.5% and 61.9% for Arm 1, 2, 3, respectively. The NSCLC cohort showed a DCR for each arm of 100% at 18 weeks and 84.2% at 48 weeks, 88.9% at 18 weeks and 72.2% at 48 weeks, and 95.2% at 18 weeks and 75% at 48 weeks for Arm 1, 2, 3, respectively. The mean progression free survival (mPFS) overall was 13.4 months and progression free survival at 12 months was 53.6%. The mPFS for each arm was 14.4 months; 12.8 months; and 10.8 months, for Arm 1, 2, 3, respectively. The progression free survival at 12 months for each arm was 67.3%, 54.5%; 40.8%, for Arm 1, 2, 3, respectively. Median OS was not reached, 1-yr OS rate over 80% (estimated) for all three arms.

### Limtied Stage SCLC (LS-SCLC)

There were a total of 33 patients enrolled in the LS-SCLC cohort. Overall, the safety findings were consistent with the known safety profiles of durvalumab±tremelimumab and cCRT. There was no evidence that addition of durvalumab±tremelimumab to standard of care chemoradiotherapy affected the tolerability of standard of care chemoradiotherapy. Specifically, patients in Arms 1 and 2 of the LS-SCLC cohort showed:
No DLTs;
Adverse events of CTCAE Grade 3 or 4 and SAE were reported for 79% and 21% of patients. The most frequent Grade 3/4 AEs were hematologic toxicities;
No fatal AEs;
2 patients had AEs leading to discontinuation of any study treatment, including durvalumab (pneumonitis, IgA nephropathy);
Oesophagitis, pneumonitis, and radiation pneumonitis events were primarily Grade 1/2 in severity. Events were manageable and one event led to treatment discontinuation; no events had a fatal outcome;
Four patients with imAEs across t2 treatment arms (pneumonitis, hypothyroid, hyperthyroid, adrenal insufficiency, dermatitis/rash). Only the pneumonitis imAE led to discontinuation. No fatal imAEs.

In SCLC Arms 3 and 4, the patients showed:
Arm 3 completed enrollment in Part A; no DLTs, however Arm 4 did not complete enrollment due to study termination. Of the 3 patients enrolled in Arm 4, no DLTs;
Adverse events of CTCAE Grade 3 or 4 were reported for 76% patients, most frequently neutropenia and anemia;
SAEs were experienced by 4 patients, 2 of them were radiation pneumonitis;
No fatal AEs;
Two patients had AEs leading to discontinuation of any study treatment including durvalumab (pneumonitis, radiation pneumonitis);
Oesophagitis, pneumonitis, and radiation pneumonitis events were primarily Grade 1/2 in severity. Events were manageable with 2 treatment discontinuations; no events had a fatal outcome;
Three patients with imAEs across the 2 treatment arms, all in Arm 3. The most common group term category was pneumonitis; only 1 of these led to discontinuation. No fatal imAEs.

In Arms 1 and 2, efficacy for the SCLC cohort showed an ORR of 67%, a median DOR of 13.4 months, a DCR of 96% at 18 weeks and 78% at 48 weeks, mPFS of 9.3 months, and PFS12 rate of 41%. A median OS was not reached and the OS12 rate was estimated 90%

In Arms 3 and 4, efficacy for the SCLC cohort showed an ORR of 89%; a DCR of 100% at 18 weeks and 100% at 48 weeks. mPFS overall and median OS were not reached. The OS12 rate was estimated 100%.

1. A method of increasing the overall response rate (ORR) or disease control rate (DCR) in a patient with small-cell lung cancer (SCLC), the method comprising concurrently treating the patient with a human anti-PD-L1 antibody, an anti-CTLA-4 antibody, and chemoradiation therapy.

2. (canceled)

3. A method of treating a patient with SCLC, the method comprising concurrently treating the patient with a human anti-PD-L1 antibody, an anti-CTLA-4 antibody, and chemoradiation therapy.

4. The method according to claim 1, wherein the SCLC is limited stage.

5. The method according to claim 1, wherein the chemoradiation therapy comprises 60 to 70 Gy in 30 to 35 fractions.

6. The method according to claim 5, wherein the chemoradiation therapy comprises 2 Gy/fraction, 5 fractions per week, over 6 to 7 weeks.

7. The method according to claim 1, wherein the chemoradiation therapy comprises hyperfractionated radiation.

8. The method according to claim 7, wherein the hyperfractionated radiation comprises 45 Gy in 30 fractions.

9. The method according to claim 8, wherein the hyperfractionated radiation comprises 1.5 Gy/fractions, 2 fractions per day at least 6 hours apart, 10 fractions per week, over 3 weeks.

10. The method according to claim 1, wherein the chemoradiation therapy comprises cisplatin and/or carboplatin and etoposide.

11. The method according to claim 1, wherein the anti-CTLA-4 antibody is tremelimumab.

12. The method according to claim 1, wherein the anti-PD-L1 antibody is durvalumab, avelumab, atezolizumab, or sugemalimab.

13. The method according to claim 1, wherein treatment with the human anti-PD-L1 antibody comprises administering 1500 mg of the anti-PD-L1 antibody to the patient intravenously every four weeks (Q4W).

14. The method according to claim 1, wherein treatment with the anti-CTLA-4 antibody comprises administering 75 mg of the anti-CTLA-4 antibody to the patient intravenously every four weeks (Q4W).

15. The method according to claim 1, wherein the anti-PD-L1 antibody and the anti-CTLA-4 antibody are administered on the first day of chemoradiation therapy.

16. A method of increasing the overall response rate (ORR) or disease control rate (DCR) in a patient with squamous cell carcinoma of the head and neck (HNSCC), the method comprising concurrently treating the patient with a human anti-PD-L1 antibody and chemoradiation therapy.

17-56. (canceled)
